(19) United States
(12) Patent Application Publication
(10) Pub. No.: US 2026/0007151 A1
(43) Pub. Date: Jan. 8, 2026

(54) BIOACTIVE BIPHASIC COMPOSITIONS

(71) Applicant: Natural Extraction Systems, LLC, Boulder, CO (US)

(72) Inventors: C. Russell Thomas, Boulder, CO (US); Douglas G. Metcalf, Boulder, CO (US)

(21) Appl. No.: 19/263,212

(22) Filed: Jul. 8, 2025

Related U.S. Application Data
(62) Division of application No. 17/392,247, filed on Aug. 2, 2021, now Pat. No. 12,349,706.
(60) Provisional application No. 63/059,829, filed on Jul. 31, 2020.
(60) Provisional application No. 63/130,560, filed on Dec. 24, 2020.
(60) Provisional application No. 63/148,047, filed on Feb. 10, 2021.
(60) Provisional application No. 63/154,480, filed on Feb. 26, 2021.
(60) Provisional application No. 63/154,507, filed on Feb. 26, 2021.
(60) Provisional application No. 63/154,538, filed on Feb. 26, 2021.
(60) Provisional application No. 63/154,579, filed on Feb. 26, 2021.
(60) Provisional application No. 63/191,814, filed on May 21, 2021.
(60) Provisional application No. 63/191,830, filed on May 21, 2021.
(60) Provisional application No. 63/191,844, filed on May 21, 2021.
(60) Provisional application No. 63/191,872, filed on May 21, 2021.
(60) Provisional application No. 63/194,810, filed on May 28, 2021.
(60) Provisional application No. 63/228,563, filed on Aug. 2, 2021.
(60) Provisional application No. 63/228,572, filed on Aug. 2, 2021.
(60) Provisional application No. 63/228,594, filed on Aug. 2, 2021.

(51) Int. Cl. A23L 2/38; A23L 2/395; A61K 9/00; A61K 31/00

(52) U.S. Cl. CPC A23L 2/38; A23L 2/395; A61K 9/0095; A61K 31/658

(57) ABSTRACT

Various aspects of this disclosure relate to excipients that comprise a solid phase and a liquid phase, wherein the solid phase has a surface-area-to-volume ratio of at least 500 per meter. In various embodiments, the liquid phase comprises an active ingredient, and the liquid phase coats substantially all of the solid phase, which increases the surface area of the liquid phase and thereby improves the bioavailability and pharmacokinetics of the active ingredient.

### CROSS-REFERENCE TO RELATED APPLICATIONS

This patent is a Division of U.S. patent application Ser. No. 17/392,247, which granted as U.S. Pat. No. 12,349,706, and which claims priority to U.S. Provisional Patent Application No. 63/059,829, filed Jul. 31, 2020; U.S. Provisional Patent Application No. 63/130,560, filed Dec. 24, 2020; U.S. Provisional Patent Application No. 63/148,047, filed Feb. 10, 2021; U.S. Provisional Patent Application No. 63/154,480, filed Feb. 26, 2021; U.S. Provisional Patent Application No. 63/154,507, filed Feb. 26, 2021; U.S. Provisional Patent Application No. 63/154,538, filed Feb. 26, 2021; U.S. Provisional Patent Application No. 63/154,579, filed Feb. 26, 2021; U.S. Provisional Patent Application No. 63/191,814, filed May 21, 2021; U.S. Provisional Patent Application No. 63/191,830, filed May 21, 2021; U.S. Provisional Patent Application No. 63/191,844, filed May 21, 2021; U.S. Provisional Patent Application No. 63/191,872, filed May 21, 2021; U.S. Provisional Patent Application No. 63/194,810, filed May 28, 2021; U.S. Provisional Patent Application No. 63/228,563, filed Aug. 2, 2021; U.S. Provisional Patent Application No. 63/228,572, filed Aug. 2, 2021; and U.S. Provisional Patent Application No. 63/228,594, filed Aug. 2, 2021, each of which is incorporated by reference in its entirety.

### BACKGROUND

The “Rule of Five” states that a druglike agent generally has a common logarithm of its octanol-water partition coefficient that is no greater than 5. A generally-applicable strategy to overcome this limitation of the Rule of Five is desirable.

### SUMMARY

Bioactive agents that possess octanol-water partition coefficients significantly greater than 1 generally display poor oral bioavailability and pharmacokinetics. Such agents favor aggregating or partitioning into a lipid phase in the gastrointestinal tract rather than assimilation into the body. The small intestine contains lipase enzymes that metabolize lipids to break apart lipid phases and release bioactive agents for absorption, but significant portions of hydrophobic agents are excreted. Upon entering the hepatic-portal circulation, bioactive agents that the body absorbs are directed into the liver where cytochrome P450 enzymes and other enzymes generally oxidize hydrophobic agents.

The detailed description that follows describes methods to disperse hydrophobic agents in biphasic compositions using excipients that readily dissolve in water. The oral administration of such biphasic compositions results in the dissolution of the excipients. When the hydrophobic agents are sufficiently separated in the biphasic composition, then the dissolution of the excipients results in the partitioning of the hydrophobic agents into the gastrointestinal epithelium rather than aggregation or partitioning into a lipid phase, which generally improves oral bioavailability and pharmacokinetics. When a biphasic composition is formulated such that dissolution of the excipients occurs in the buccal cavity, for example, then oral administration can bypass the hepatic-portal circulation to further improve oral bioavailability and pharmacokinetics.

### DETAILED DESCRIPTION

Various aspects of this disclosure relate to a composition, comprising a solid phase and a liquid phase, wherein the solid phase has a surface-area-to-volume ratio that is greater than 500 per meter, and the composition comprises the solid phase at a greater concentration by mass than the liquid phase. Without limiting this disclosure or any claim that matures from this patent document, increasing the surface area of the solid phase increases the surface area of the liquid phase, which accelerates dissolution of the composition following consumption.

In some embodiments, the solid phase consists of ingredients; the ingredients comprise carbohydrates; at least 95 percent of the ingredients of the solid phase consist of the carbohydrates by mass; and the carbohydrates consist of sugar alcohols. In some specific embodiments, the solid phase consists of ingredients, and at least 95 percent of the ingredients of the solid phase consist of xylitol. Without limiting this disclosure or any claim that matures from this patent document, carbohydrates are generally soluble in water, which allows rapid dissolution following consumption; sugar alcohols display robust stability across a broad pH range; and xylitol displayed superior performance relative to other commercially-available sugar alcohols during research and development of the disclosed compositions.

In some embodiments, the composition comprises the liquid phase at a concentration of at least 0.1 percent and no greater than 35 percent by mass; and the composition comprises the solid phase at a concentration of at least 65 percent and no greater than 99.9 percent by mass.

In some embodiments, the liquid phase comprises at least 3 and no greater than 8 calories of food energy per gram of the liquid phase; the solid phase comprises at least 0.1 and no greater than 4.5 calories of food energy per gram of the solid phase; the liquid phase comprises at least 0.02 and no greater than 1 calories of food energy per gram of the composition; and the solid phase comprises at least 0.1 and no greater than 3 calories of food energy per gram of the composition. “Food calorie” refers to a North American food calorie, which is equal to a European Union food kilocalorie, and which are both equal to 4.184 kilojoules of food energy.

In some embodiments, the liquid phase consists of ingredients that comprise a solvent; each of the ingredients of the liquid phase has a concentration by mass in the liquid phase; and the concentration by mass of the solvent in the liquid phase is greater than the concentration by mass of any of the other ingredients of the liquid phase. In some specific embodiments, the solvent has a solubility in water at 21 degrees Celsius of at least 10 grams per liter. In some very specific embodiments, the solvent is ethanol, propane-1,2-diol, or propane-1,2,3-triol. Without limiting this disclosure or any claim that matures from this patent document, the solvent should be selected to dissolve a high concentration of the active ingredient without dissolving a significant portion of the solid phase, and the solvent should have a high solubility in water.

In some embodiments, the liquid phase comprises ethanol and ethoxide at a molar ratio of at least 100:1 and no greater than 100,000:1. In some embodiments, the composition comprises water and hydroxide, wherein the water and the hydroxide are solutes that are dissolved in the solvent of the liquid phase at a molar ratio of at least 100:1 and no greater than 100,000:1.

In some embodiments, the composition a cation, wherein the cation is a solute that is dissolved in the solvent of the liquid phase. In some specific embodiments, the cation is sodium cation or potassium cation. In some specific embodiments, the cation is dissolved in the solvent of the liquid phase at a concentration of at least 100 micromolar and no greater than 500 millimolar.

In some embodiments, the ingredients comprise an active ingredient; and the active ingredient is a solute that is dissolved in the solvent of the liquid phase.

In some embodiments, the active ingredient is an anion. In some specific embodiments, the active ingredient has a charge of-1.

In some embodiments, the active ingredient is dissolved in the solvent of the liquid phase at a concentration of at least 1 millimolar and no greater than 500 millimolar.

In some embodiments, the active ingredient has a solubility in water at 37 degrees Celsius; and the active ingredient is dissolved in the solvent of the liquid phase at a concentration that is greater than the solubility of the active ingredient in water at 37 degrees Celsius.

In some embodiments, the anion is a conjugate base of a molecule that has an acid dissociation constant in water at 37 degrees Celsius of at least 50 femtomolar and no greater than 50 nanomolar for conversion of the molecule into the active ingredient. In some embodiments, the composition comprises the active ingredient and the molecule at a molar ratio of at least 1:1 and no greater than 10,000:1. In some embodiments, the molecule has an octanol-water partition coefficient of at least 10 and no greater than 10,000,000.

In some embodiments, the active ingredient is 2-geranyl-3-hydroxy-5-pentylphenolate; 3-hydroxy-2-(6-isopropenyl-3-methylcyclohex-2-enyl)-5-pentylphenolate; 3-hydroxy-2-(6-isopropenyl-3-methylcyclohex-3-enyl)-5-pentylphenolate; 6,6,9-trimethyl-3-pentyl-6a,7,8,10a-tetrahydro-6H-benzo[c]chromene-1-oxide; 6,6,9-trimethyl-3-pentyl-6a, 7, 10, 10a-tetrahydro-6H-benzo[c]chromene-1-oxide; 6,6,9-trimethyl-3-pentyl-6H-benzo[c]chromene-1-oxide; or a variant of any one of the foregoing in which pentyl is replaced with an alkyl group such as propyl as further defined in embodiment 1200.

Various aspects of this disclosure relate to a composition, comprising a solid phase and a liquid phase, wherein the composition is formulated for oral administration to a human; the liquid phase comprises at least 3 and no greater than 5 calories of food energy per gram of the liquid phase; the solid phase has a surface-area-to-volume ratio that is greater than 500 per meter; the liquid phase coats at least 80 percent of the surface area of the composition; and the composition comprises the solid phase at a greater concentration by mass than the liquid phase.

In some embodiments, the solid phase consists of ingredients; and at least 50 percent by mass of the ingredients of the solid phase have solubilities in water at 21 degrees Celsius of at least 10 grams per liter.

In some embodiments, the ingredients of the solid phase comprise carbohydrates. In some embodiments, each carbohydrate of the carbohydrates has the chemical formula CxHyOz, wherein x is an integer greater than 3 and no greater than 12; y is an integer greater than 1.8× and no greater than 2x+2; and z is an integer of at least 0.8× and no greater than x.

In some embodiments, the carbohydrates comprise pentane-1,2,3,4,5-pentol.

In some embodiments, at least 80 percent of the ingredients of the solid phase consist of the carbohydrates by mass.

In some embodiments, the composition comprises the liquid phase at a concentration of at least 0.5 percent and no greater than 15 percent by mass; and the composition comprises the solid phase at a concentration of at least 85 percent and no greater than 99.5 percent by mass.

In some embodiments, the solid phase comprises at least 0.2 and no greater than 3.5 calories of food energy per gram of the solid phase; the liquid phase comprises at least 0.05 and no greater than 0.5 calories of food energy per gram of the composition; and the solid phase comprises at least 0.1 and no greater than 3 calories of food energy per gram of the composition.

In some embodiments, the liquid phase consists of ingredients that comprise a solvent; the solvent is miscible with water; each of the ingredients of the liquid phase has a concentration by mass in the liquid phase; and the concentration by mass of the solvent in the liquid phase is greater than the concentration by mass of any of the other ingredients of the liquid phase.

In some embodiments, the solvent is propane-1,2-diol.

In some embodiments, the ingredients of the liquid phase comprise a cosolvent; the cosolvent is ethanol; and the solvent and the cosolvent are different ingredients.

In some embodiments, the liquid phase comprises propane-1,2-diol and one or both of 1-hydroxypropane-2-oxide and 2-hydroxypropane-1-oxide at a molar ratio of at least 100:1 and no greater than 10,000,000:1 (propane-1,2-diol: 1-hydroxypropane-2-oxide and 2-hydroxypropane-1-oxide); and the liquid phase comprises ethanol and ethoxide at a molar ratio of at least 100:1 and no greater than 10,000,000:1.

In some embodiments, the composition comprises a cation, wherein the cation is potassium cation (“K+”); and the cation is a solute that is dissolved in the solvent of the liquid phase.

In some embodiments, the ingredients of the liquid phase comprise an active ingredient; and the active ingredient is a solute that is dissolved in the solvent of the liquid phase.

In some embodiments, the active ingredient has a solubility in water at 37 degrees Celsius; and the active ingredient is dissolved in the solvent of the liquid phase at a concentration that is at least 100 percent greater than the solubility of the active ingredient in water at 37 degrees Celsius.

In some embodiments, the active ingredient has an octanol-water partition coefficient of at least 10.

In some embodiments, the active ingredient is a molecule; and the molecule is 11-methoxy-12-hydroxydehydrokavain.

In some embodiments, the active ingredient is a molecule; and the molecule is 5-hydroxykavain.

In some embodiments, the composition comprises cinnamaldehyde, which masks the flavor of kavalactones.

Nothing in the foregoing disclosure shall limit any patent claim that matures from this disclosure, which claim(s) shall instead be construed according to the language of the claim(s) in the context of their claim dependencies.

1. A composition, comprising a solid phase and a liquid phase, wherein:
the composition is formulated for oral administration to a human;
the liquid phase comprises at least 3 and no greater than 5 calories of food energy per gram of the liquid phase;
the solid phase has a surface-area-to-volume ratio that is greater than 500 per meter;
the liquid phase coats at least 80 percent of the surface area of the composition; and
the composition comprises the solid phase at a greater concentration by mass than the liquid phase.

2. The composition of claim 1, wherein:
the solid phase consists of ingredients; and
at least 50 percent by mass of the ingredients of the solid phase have solubilities in water at 21 degrees Celsius of at least 10 grams per liter.

3-21. (canceled)

22. The composition of claim 1, wherein the ingredients of the solid phase comprise carbohydrates.

23-25. (canceled)

26. The composition of claim 22, wherein:
each carbohydrate of the carbohydrates has the chemical formula CxHyOz;
x is an integer greater than 3 and no greater than 12;
y is an integer greater than 1.8× and no greater than 2x+2; and
z is an integer of at least 0.8× and no greater than x.

27-103. (canceled)

104. The composition of claim 22, wherein the carbohydrates comprise pentane-1,2,3,4,5-pentol.

105-187. (canceled)

188. The composition of claim 22, wherein at least 80 percent of the ingredients of the solid phase consist of the carbohydrates by mass.

189-225. (canceled)

226. The composition of claim 1, wherein:
the composition comprises the liquid phase at a concentration of at least 0.5 percent and no greater than 15 percent by mass; and
the composition comprises the solid phase at a concentration of at least 85 percent and no greater than 99.5 percent by mass.

227-314. (canceled)

315. The composition of claim 1 , wherein:
the solid phase comprises at least 0.2 and no greater than 3.5 calories of food energy per gram of the solid phase;
the liquid phase comprises at least 0.05 and no greater than 0.5 calories of food energy per gram of the composition; and
the solid phase comprises at least 0.1 and no greater than 3 calories of food energy per gram of the composition.

316-321. (canceled)

322. The composition of claim 1, wherein:
the liquid phase consists of ingredients that comprise a solvent;
the solvent is miscible with water;
each of the ingredients of the liquid phase has a concentration by mass in the liquid phase; and
the concentration by mass of the solvent in the liquid phase is greater than the concentration by mass of any of the other ingredients of the liquid phase.

323-336. (canceled)

337. The composition of claim 322, wherein the solvent is propane-1,2-diol.

338-339. (canceled)

340. The composition of claim 322, wherein:
the ingredients of the liquid phase comprise a cosolvent;
the cosolvent is ethanol; and
the solvent and the cosolvent are different ingredients.

341-360. (canceled)

361. The composition of claim 1 359 or 360, wherein:
the liquid phase comprises propane-1,2-diol and one or both of 1-hydroxypropane-2-oxide and 2-hydroxypropane-1-oxide at a molar ratio of at least 100:1 and no greater than 10,000,000:1 (propane-1,2-diol: 1-hydroxypropane-2-oxide and 2-hydroxypropane-1-oxide); and
the liquid phase comprises ethanol and ethoxide at a molar ratio of at least 100:1 and no greater than 10,000,000:1.

362-372. (canceled)

373. The composition of claim 322, comprising a cation, wherein:
the cation is potassium cation (“K+”); and
the cation is a solute that is dissolved in the solvent of the liquid phase.

374-412. (canceled)

413. The composition of claim 322, wherein:
the ingredients of the liquid phase comprise an active ingredient; and
the active ingredient is a solute that is dissolved in the solvent of the liquid phase.

414-416. (canceled)

417. The composition of claim 413, wherein:
the active ingredient has a solubility in water at 37 degrees Celsius; and
the active ingredient is dissolved in the solvent of the liquid phase at a concentration that is at least 100 percent greater than the solubility of the active ingredient in water at 37 degrees Celsius.

418-420. (canceled)

421. The composition of claim 413, wherein the active ingredient has an octanol-water partition coefficient of at least 10.

422-484. (canceled)

485. The composition of claim 413, wherein;
the active ingredient is a molecule; and
the molecule is 11-methoxy-12-hydroxydehydrokavain.

486-487. (canceled)

488. The composition of claim 413, wherein;
the active ingredient is a molecule; and
the molecule is 5-hydroxykavain.

489-490. (canceled)

491. The composition of claim 1, comprising cinnamaldehyde.

492-516. (canceled)

517. A method to prepare a beverage formulation, comprising:
providing a composition comprising an active ingredient;
providing a beverage; and
combining the composition with the beverage to disperse the active ingredient in the beverage and to prepare the beverage formulation,
wherein:
the composition comprises a solid phase and a liquid phase;
the solid phase has a surface-area-to-volume ratio that is greater than 500 per meter;
the solid phase consists of ingredients;
the ingredients of the solid phase comprise carbohydrates;
at least 90 percent of the ingredients of the solid phase consist of the carbohydrates by mass;
the composition comprises the liquid phase at a concentration of at least 0.5 percent and no greater than 15 percent by mass;
the composition comprises the solid phase at a concentration of at least 85 percent and no greater than 99.5 percent by mass;
the composition comprises at least 0.5 and no greater than 5 calories of food energy per gram of the composition;
the liquid phase comprises a concentration of calories of food energy per gram of the liquid phase;
the solid phase comprises a concentration of calories of food energy per gram of the solid phase that is less than the concentration of calories of food energy per gram of the liquid phase;
the liquid phase consists of ingredients that comprise a solvent;
each of the ingredients of the liquid phase has a concentration by mass in the liquid phase;
the concentration by mass of the solvent in the liquid phase is greater than the concentration by mass of any of the other ingredients of the liquid phase;
the solvent is propane-1,2-diol;
the ingredients of the liquid phase comprise the active ingredient;
the active ingredient is a solute that is dissolved in the solvent of the liquid phase;
the active ingredient is dissolved in the solvent of the liquid phase at a concentration of at least 200 micromolar and no greater than 250 millimolar;
the active ingredient has a solubility in water at 37 degrees Celsius;
the active ingredient is dissolved in the solvent of the liquid phase at a concentration that is greater than the solubility of the active ingredient in water at 37 degrees Celsius;
the active ingredient has an octanol-water partition coefficient of at least 10; and
the beverage is a liquid that comprises water at a concentration of at least 50 molar.
